(19) United States
(12) Patent Application Publication
(10) Pub. No.: US 2011/0293595 A1
(43) Pub. Date: Dec. 1, 2011

(54) Prophylactic and therapeutic treatment of Alzheimer's Disease, neuro-degenerative diseases, protein aggregation diseases, Parkinson's Disease and amyloid diseases, using phytic acid and phytate

(76) Inventor: Robert Sabin, Mill Neck, NY (US)

(21) Appl. No.: 13/135,909

(22) Filed: Aug. 9, 2011

Related U.S. Application Data
(63) Continuation-in-part of application No. 12/924,407, filed on Sep. 27, 2010.
Application No. 12/924,407 is a continuation-in-part of application No. 12/661,964, filed on Mar. 26, 2010.

(30) Foreign Application Priority Data
Mar. 26, 2010 (US) PCT/US2010/000927
Sep. 27, 2010 (US) PCT/US2010/002624

(51) Int. Cl. A61K 38/46; A61P 25/28; A61P 25/16; A61P 9/10; A61P 3/10; A61P 31/00; A61P 29/00; A61K 31/6615; A61P 25/00

(52) U.S. Cl. 424/94.6; 514/103

(57) ABSTRACT

A composition and method for the treatment of Alzheimer's Disease, and/or related protein aggregation diseases and/or amyloidoses and/or a neuro-degenerative disease, and/or Parkinson's Disease and/or Parkinsonism including an effective amount of a compound selected from the group consisting of phytic acid (inositol hexakisphosphate), a phytate salt, an isomer or hydrolysate of phytic acid or a phytate salt, or a mixture of any combination thereof, being administered to a person in an amount from about 0.5 grams to about 18.75 grams at least once a week up to once per day, with or without a dephosphorylating enzyme. The dosage may be proportionately or disproportionately divided so that the dosage is administered in proportionally or disproportionately reduced amounts which cumulatively add up to a predetermined dosage, in a time period where the proportionately or disproportionately divided dosages are cumulatively equivalent in quantity to the dosage.

### RELATED APPLICATIONS

This application is a continuation-in-part of application Ser. No. 12/924,407 filed Sep. 27, 2010, which is a continuation-in-part of application Serial No. 12/661,964 filed Mar. 26, 2010 and claims priority from under 35 U.S.C. 120 therefrom. This application is also based in part upon provisional application Nos. 61/273,126 filed Jul. 29, 2009 and 61/211,230 filed Mar. 27, 2009 and provisional application No. 61/273,126, filed Jul. 29, 2009 and claims benefit under 35 U.S.C. 119(e) and priority therefrom. This application also claims priority under 35 U.S.C. §119(e). from Patent Cooperation Treaty foreign application number PCT/US2010/000927 filed on Mar. 26, 2010. These applications are incorporated by reference herein.

All Patents, Scientific Articles, and other Documents mentioned herein are incorporated by reference as if reproduced in full below.

### FIELD OF THE INVENTION

This invention relates to the prophylactic and therapeutic treatment of Alzheimer's Disease, herein below identified as “AD”, and/or protein aggregation diseases and/or amyloidoses and/or a neuro-degenerative disease, and/or Parkinson's Disease and/or Parkinsonism.

### BACKGROUND OF THE INVENTION

Phytic acid, generally accepted as having the structure myo-inositol-hexakis (dihydrogen phosphate), is a major component of plant seeds, constituting 1-3% by weight of many cereals and oil seeds. Most wheat brans contain between 4 and 5% phytic acid. Phytic acid may be prepared in pure form from various plant sources, such as wheat, corn, soybeans, sesame seeds, peanuts, lima beans, barley, oats, wild rice and sunflower seeds. It can be extracted with dilute hydrochloric acid at room temperature, precipitated with various reagents including ferric chloride, bicarbonates, potassium hydroxide, sodium hydroxide, ammonium hydroxide, calcium hydroxide, magnesium hydroxide or alcohol. It is then further purified by conventional chemical techniques.

When one or more of the acidic protons of the phosphate groups in phytic acid are replaced by a counterion, the compound is usually referred to as a phytate salt. The special name phytin is used for the calcium-magnesium salt of phytate derived from plant seeds (a product of Ciba-Geigy). Also, while phytic acid contains six phosphate groups, when introduced into the digestive tract of an animal, one or more of the phosphate groups may be hydrolyzed by the action of the digestive acids and enzymes. The main uses of phytic acid include use as a food additive for preservation of foods. Studies on the use of phytic acid as a food additive show that ingestion of large doses of phytic acid elicits no physiological discomfort or symptoms of any toxicological action in humans. See Starkenstein, Biochem. Z. 30: 56 (1911). Phytic acid and its metabolites are thus not believed to be toxic or highly reactive.

Phytic acid is the 6 phosphates ester of inositol. Inositol, chemically hexahydroxycylohexane, is any of nine stereoisomeric alcohols that closely resemble glucose in structure. It is a constituent of many cell phosphoglycerides. Meso- or myo-inositol, named for its presence in muscle tissue, is biologically the important isomer. Myo-inositol is the precursor in the phosphatidylinositol cycle, a source of two second messengers (diacylglycerol and inositol triphosphate). Inositols and their phosphates lack a hydrolytically labile glycosidic linkage and are stable to degradative enzymes in vivo. They have been used in the stable insulin mediators, inhibitors, and modulators. The phytic acid may be in a salt form, such as, for example, calcium magnesium phytate salt.

Two reports from Science Magazine of 3 Nov. 2006 support the importance of reducing Abeta plaque production as a therapeutic goal. See Goedert and Spillantini “A Century of Alzheimer's Disease”, SCIENCE, Vol. 314, 3 November 2006, pp 779-781. See also Roberson and Mucke, “100 Years and Counting: Proposals for Defeating Alzheimer's Disease,” SCIENCE, Vol. 314, 3 Nov. 2006, pp. 781-784.

U.S. Pat. No. 4,847,082 (referred to herein as the '082 patent) and U.S. Pat. No. 4,758,430 (referred to herein as the '430 patent) both to Sabin, each of which is expressly incorporated herein by reference, disclose one embodiment for administration of phytic acid or equivalent phytate salt, isomer or hydrolysate as about one-half to three grams per kilogram of body weight orally per day.

Scyllo inositol, has been shown to prevent and reverse Alzheimer's Disease in a transgenic mouse model of Alzheimer's Disease (See Nature Medicine article) (See “A Sweet Solution to Alzheimer's Disease”) (See “ALD103 May Prevent and Reverse Alzheimer's Disease”) (See “Scyllo-Inositol Appears Promising for Alzheimer's Disease”). Scyllo inositol also inhibits and blocks the aggregation of amyloid-B peptide (AB) in these same transgenic Alzheimer Disease mouse studies. The '082 patent discloses scyllo inositol as an isomeric form of phytic acid. Scyllo inositol is also disclosed in U.S. patent application Ser. No. 10/787,621 of McLaurin. This application discloses a litany of compounds for the treatment of disorders of protein aggregation, the most preferred compound of which is scyllo inositol.

### SUMMARY OF THE INVENTION

A composition including a compound selected from the group including phytic acid (inositol hexakisphosphate), phytate salt, an isomer or hydrolysate of phytic acid or a phytate salt, or a mixture of any combination thereof, with or without a dephosphorylating enzyme, such as phytase, is administered as a treatment for prevention and treatment of Alzheimer's Disease, herein below identified as “AD”, and/or protein aggregation diseases and/or amyloidoses and/or a neuro-degenerative disease, and/or Parkinson's Disease and/or Parkinsonism.

The composition lowers amyloid, which is implicated in a multitude of diseases.

In addition to the treatment and/or prevention of Alzheimer's disease and the treatment of protein aggregation diseases, such as the aggregation of beta amyloid proteins and tau protein in the brain, amyloidoses, including AL amyloidosis, amyloid A (AA) reactive systemic amyloidosis, amyloid trans-thyretin (ATTR), and accumulation of islet amyloid polypeptide type 2 diabetes, one or more neuro-degenerative diseases and protein aggregation diseases that are treated and/or prevented using the present composition include multiple sclerosis, conditions of the central or peripheral nervous system or systemic organ associated with a disorder in protein folding or aggregation, or amyloid formation, deposition, accumulation, or persistence; abnormal protein folding, abnormal protein aggregation, amyloid formation, deposition, accumulation, or persistence, or amyloid lipid interactions conditions causing the dissociation of abnormally aggregated proteins and/or dissolving or disrupting pre-formed or pre-deposited amyloid fibril or amyloid in a subject; conditions of the central or peripheral nervous system or systemic organ resulting in the deposition of proteins, protein fragments and peptides in beta-pleated sheats and/or fibrils and/or aggregates; amyloid angiopathy; mild cognitive impairment; Alzheimer's disease-related dementia; tauopathy; alpha-synucleinopathy; Amyotrophic Lateral Sclerosis; motor neuron disease; Huntington's Disease, spinocerebellar ataxia, Freidrich's Ataxia; neuro degenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy; Inclusion Body myositis due to amyloid beta peptide; Familial and Finnish Type Amyloidosis; Systemic amyloidosis associated with multiple myeloma; Familial Mediterranean Fever; chronic infections and inflammations; Type II Diabetes Mellitus associate with islet amyloid polypeptide (IAPP); vascular caused Alzheimer's Disease, Alzheimer dementia and tauopathy selected from the group of argyrophilic grain dementia, corticobasal degeneration, dementia pugilistica, diffuse neurofibrillary tangles with calcification, frontotemporal dementia with Parkinsonism, Hallervorden-Spatz disease, myotonic dystrophy, Niemann-Pick disease type C, non-Guamanian Motor Neuron disease with neurofibrillary tangles, Pick's disease, postencephalitic Parkinsonism, prion protein cerebral amyloid angiopathy, progressive subcortical gliosis, progressive supranuclear palsy, subacute sclerosing panencephalitis, tangle only dementia; alpha-synucleinopathy selected from the group of dementia with Lewy bodies, multiple system atrophy with glial cytoplasmic inclusions, Shy-Drager syndrome, striatonigral degeneration, olivopontocerebellar atrophy, neuro-degeneration with brain iron accumulation type I, olfactory dysfunction, motor neuron disease associated with filaments and aggregates of neurofilament and/or superoxide dismutase proteins, spastic paraplegia associated with defective function of chaperones and/or triple A proteins and spinocerebellar ataxia including DRPLA or Machado-Joseph Disease; Prion related disease selected from the group of Creutzfeldt-Jakob disease, Kuru, fatal familial insomnia, Gerstmann-Straussler-Scheinker disease, variant Creutzfeldt-Jakob disease, as well as amyloid polyneuropathy including senile amyloid polyneuropathy or systemic amyloidoses, Parkinson's Disease, Parkinsonism, atherosclerosis and arteriosclerosis.

The present invention also includes the use of hydrolysates of phytic acid and phytate salts wherein one or more of the phosphate groups have been removed.

A pharmaceutically effective yet surprisingly low dose of the compounds phytic acid (inositol hexakisphosphate), phytate salt, an isomer or hydrolysate of phytic acid or a phytate salt, or a mixture of any combination thereof, except for scyllo inositol, with or without a dephosphorylating enzyme, such as phytase, are used as a prophylactic and therapeutic treatment for Alzheimer's Disease, and/or protein aggregation diseases and/or amyloidoses, including AL amyloidosis, amyloid A (AA) reactive systemic amyloidosis, amyloid trans-thyretin (ATTR) and accumulation of islet amyloid polypeptide type 2 diabetes, a neuro-degenerative disease, and/or Parkinson's Disease and/or Parkinsonism. In a preferred embodiment, a dose of about 0.5 gram to about 18.75 gms of phytic acid, phytate salt, an isomer or hydrolysate of phytic acid or a phytate salt, or mixture of any combination thereof, except for scyllo inositol, are adminstered with or without a dephosphorylating enzyme, such as phytase, are administered orally per day. This dose is 1/75th to one half (½) of the previously disclosed doses in the Sabin patents cited above. Phytate salt may also be administered as a capsule orally and transdermally, and the calcium magnesium phytate salt, as packaged by many suppliers such as Jarrow Formulations, is desirable.

Surprisingly, it has been discovered that very low doses of phytic acid (inositol hexakisphosphate), phytate salt, an isomer or hydrolysate of phytic acid or a phytate salt, or a mixture of any combination thereof are pharmaceutically effective in the treatment and prevention of aggregation of beta amyloid proteins as are commonly seen in the brains of Alzheimer's patients. Treatment with such a low dose of phytic acid (inositol hexakisphosphate), phytate salt, an isomer or hydrolysate of phytic acid or a phytate salt, or a mixture of any combination thereof prevents the occurrence of potential side effects observed with high dose treatments and allows for ease of administration to Alzheimer's patients who may find it difficult to take high doses of the substance. Furthermore, it has been discovered that phytic acid, phytate salt, an isomer or hydrolysate of phytic acid or a phytate salt, or a mixture of any combination thereof is much more effective than scyllo inositol and myo inositol.

In one embodiment there is disclosed a composition for the treatment of Alzheimer's Disease and/or protein aggregation diseases and/or amyloidoses, including AL amyloidosis, amyloid A (AA) reactive systemic amyloidosis, amyloid trans-thyretin (ATTR), accumulation of islet amyloid polypeptide type 2 diabetes, and/or a neuro-degenerative disease, and/or Parkinson's Disease and/or Parkinsonism, by providing a person in need thereof with an a pharmaceutically effective amount of phytic acid (inositol hexakisphosphate), phytate salt, an isomer or hydrolysate of phytic acid or a phytate salt, or a mixture of any combination thereof, with or without a dephosphorylating enzyme, such as phytase.

In another embodiment, there is disclosed a method for the treatment of Alzheimer's Disease and/or protein aggregation diseases and/or amyloidoses, including AL amyloidosis, amyloid A (AA) reactive systemic amyloidosis, amyloid trans-thyretin (ATTR), accumulation of islet amyloid polypeptide type 2 diabetes, and/or a neuro-degenerative disease, and/or Parkinson's Disease and/or Parkinsonism. by the oral administration of 0.5 to 18.75 grams of phytic acid, phytate salt, an isomer or hydrolysate of phytic acid or a (inositol hexakisphosphate), phytate salt, an isomer or hydrolysate of phytic acid or a phytate salt, or a mixture of any combination thereof, with or without a dephosphorylating enzyme, such as phytase, per day. Alternatively, the compounds of the invention may be administered topically as a cream, ointment, gel and the like, transdermally or intradermally.

In an alternate embodiment of about 0.5 to about 18.73 grams of phytic acid administered, dosage is at least once a week. In an alternate embodiment, dosage is every other day. In a preferred embodiment, dosage is 5 days administration, followed by 2 days off. It should also be understood that the dosage of once per day can mean divided doses throughout the duration of the day. For example, if the dosage is 10 gms, once a day, the dosage can be proportionately or disproportionately divided so that the 10 gms of IP6 could be taken at a dosage of 3.33 gms three times per day or at a dosage of 4 gms, 3 gms and 3 gms per day, adding up to the daily cumulative dosage of 10 gms once a day. These divided doses during the day remain equivalent in quantity to a dosage of 10 gm once a day.

For a better understanding of the present invention, together with other and further objects, reference is made to the following description, taken in conjunction with the examples, and its scope will be pointed out in the appended claims.

### BRIEF DESCRIPTION OF THE DRAWINGS

The features and advantages of the present invention will become apparent from the following detailed description of a preferred embodiment thereof, taken in conjunction with the accompanying drawings, in which:

FIG. 1 is a chart of cell viability.

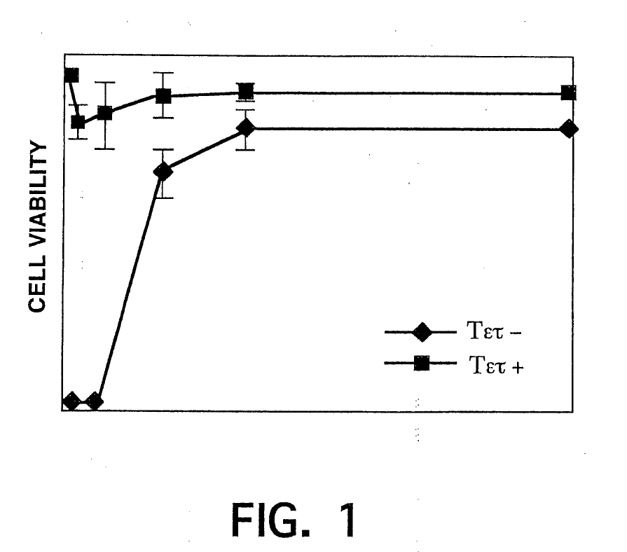

FIG. 2 is a chart of a bar graph of the percentage of H₂O₂, where V=control and PA=phytic acid.

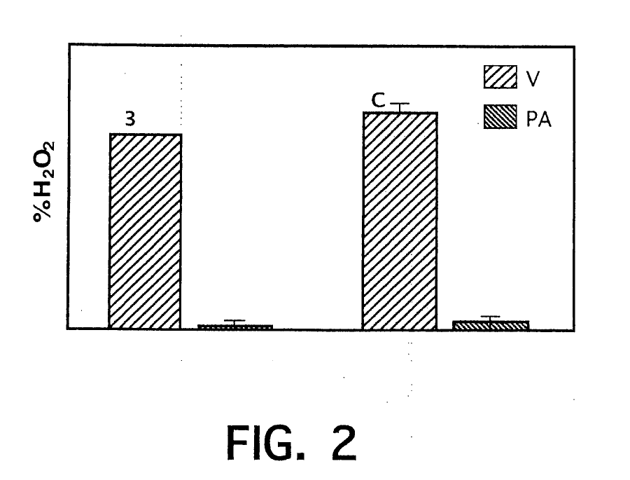

FIG. 3 is a chart of cell viability of scyllo inositol and myo inositol.

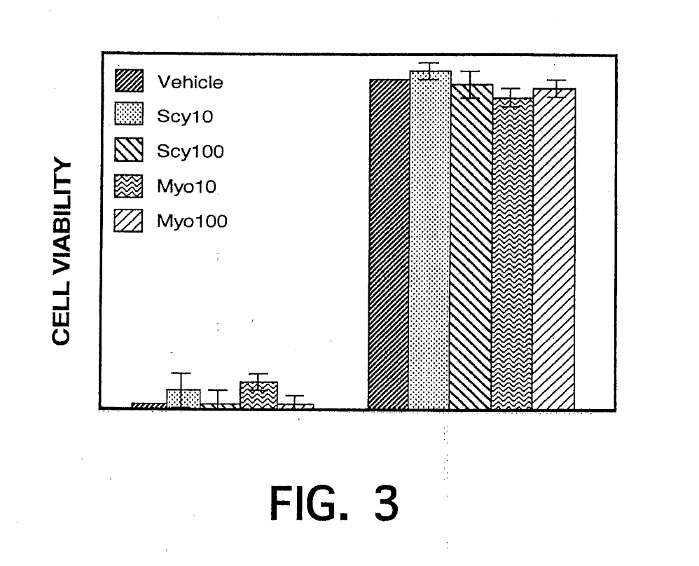

FIG. 4 is a chart of soluble amyloid beta 1-42/μgug protein, phytic acid and a vehicle carrier.

FIG. 5 is a chart of soluble amyloid beta-40/μgug protein, phytic acid and a vehicle carrier.

FIG. 6 is a chart of insoluble amyloid beta 1-42/μgug protein, phytic acid and vehicle carrier.

### DETAILED DESCRIPTION

The method according to the present invention comprises treating a subject, afflicted with Alzheimer's Disease, herein below identified as “AD”, and/or protein aggregation diseases and/or amyloidoses, including AL amyloidosis, amyloid A (AA) reactive systemic amyloidosis, amyloid trans-thyretin (ATTR), accumulation of islet amyloid polypeptide type 2 diabetes, and/or a neuro-degenerative disease, and/or Parkinson's Disease and/or Parkinsonism, with a composition in which the active ingredient is a compound selected from the group including phytic acid (inositol hexakisphosphate), a phytate salt, or an isomer or hydrolysate of phytic acid or phytate salt, with or without a dephosphorylating enzyme, such as described in U.S. Pat. No. 5,206,226 of Sabin, herein incorporated by reference in its entirety. In one embodiment of the present invention, the dephosphorylating enzyme is phytase.

The present composition provides a treatment for prevention and/or treatment of one or more diseases including Alzheimer's Disease, and/or protein aggregation diseases and/or amyloidoses, including AL amyloidosis, amyloid A (AA) reactive systemic amyloidosis, amyloid trans-thyretin (ATTR), accumulation of islet amyloid polypeptide type 2 diabetes, and/or a neuro-degenerative disease, and/or Parkinson's Disease and/or Parkinsonism. In addition to the treatment and/or prevention of Alzheimer's disease and the treatment of protein aggregation diseases such as the aggregation of beta amyloid proteins and tau protein in the brain, one or more neuro-degenerative diseases and protein aggregation diseases that are treated and/or prevented using the present composition include multiple sclerosis, conditions of the central or peripheral nervous system or systemic organ associated with a disorder in protein folding or aggregation, or amyloid formation, deposition, accumulation, or persistence; abnormal protein folding, abnormal protein aggregation, amyloid formation, deposition, accumulation, or persistence, or amyloid lipid interactions conditions causing the dissociation of abnormally aggregated proteins and/or dissolving or disrupting pre-formed or pre-deposited amyloid fibril or amyloid in a subject; conditions of the central or peripheral nervous system or systemic organ resulting in the deposition of proteins, protein fragments and peptides in beta-pleated sheats and/or fibrils and/or aggregates; amyloid angiopathy; mild cognitive impairment; Alzheimer's disease-related dementia; tauopathy; alpha-synucleinopathy; Amyotrophic Lateral Sclerosis; motor neuron disease; Huntington's Disease, spinocerebellar ataxia, Freidrich's Ataxia; neuro degenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy; Inclusion Body myositis due to amyloid beta peptide; Familial and Finnish Type Amyloidosis; Systemic amyloidosis associated with multiple myeloma; Familial Mediterranean Fever; chronic infections and inflammations; Type II Diabetes Mellitus associate with islet amyloid polypeptide (IAPP); vascular caused Alzheimer's Disease, Alzheimer dementia and tauopathy selected from the group of argyrophilic grain dementia, corticobasal degeneration, dementia pugilistica, diffuse neurofibrillary tangles with calcification, frontotemporal dementia with Parkinsonism, Hallervorden-Spatz disease, myotonic dystrophy, Niemann-Pick disease type C, non-Guamanian Motor Neuron disease with neurofibrillary tangles, Pick's disease, postencephalitic Parkinsonism, prion protein cerebral amyloid angiopathy, progressive subcortical gliosis, progressive supranuclear palsy, subacute sclerosing panencephalitis, tangle only dementia; alpha-synucleinopathy selected from the group of dementia with Lewy bodies, multiple system atrophy with glial cytoplasmic inclusions, Shy-Drager syndrome, striatonigral degeneration, olivopontocerebellar atrophy, neuro-degeneration with brain iron accumulation type I, olfactory dysfunction, motor neuron disease associated with filaments and aggregates of neurofilament and/or superoxide dismutase proteins, spastic paraplegia associated with defective function of chaperones and/or triple A proteins and spinocerebellar ataxia including DRPLA or Machado-Joseph Disease; Prion related disease selected from the group of Creutzfeldt-Jakob disease, Kuru, fatal familial insomnia, Gerstmann-Straussler-Scheinker disease, and variant Creutzfeldt-Jakob disease, as well as amyloid polyneuropathy including senile amyloid polyneuropathy or systemicamyloidoses, Parkinson's Disease, Parkinsonism, atherosclerosis and arteriosclerosis.

By the term isomer as used herein, it is intended to include the various conformations of phytic acid (inositol hexakisphosphate), as described hereinabove, and the corresponding conformations of phytate salts with or without a dephosphorylating enzyme, such as phytase. The term salts is broadly intended to cover any of the various salts formed by the replacement of any or all of the available acidic protons of the phosphate groups with a counterion. The counterion may be any pharmaceutically acceptable counterion such as sodium, magnesium, potassium, zinc, ferric, ferrous, and the like, including organic counterions such as quaternary ammonium ions and ions of organic bases.

The present invention also includes the hydrolysates of phytic acid (inositol hexakisphosphate) and phytate salts wherein one or more of the phosphate groups have been removed with or without a dephosphorylating enzyme, such as phytase. Once administered into the digestive tract, bloodstream, the phytic acid or phytate salt may be hydrolyzed by digestive, blood or cellular enzymes, thereby removing one or more of the phosphate groups on the cyclohexane ring. However, it is contemplated to be within the scope of the invention that these hydrolysates of phytic acid and phytate salts, with or without a dephosphorylating enzyme, may also be administered directly to the subject and therefore are within the scope of the present invention.

The hydrolysates of phytic acid (inositol hexakisphosphate) and phytate salts may be prepared by partial acid or basic hydrolysis or by hydrolysis using enzymes prior to preparation of dosage forms for administration. Preferably, the hydrolysates will be made in vivo by co-administering with phytic acid or phytate salt an enzyme which hydrolyzes phosphate groups, such as 3-phytase, 6-phytase or acid phosphatase, with or without a dephosphorylating enzyme.

The phytic acid (inositol hexakisphosphate) or phytate salt may be absorbed into or adsorbed onto a solid carrier to facilitate pharmaceutical administration. For example, phytic acid may be formulated into a starch powder by spray drying or vacuum drying an aqueous mixture of phytic acid and dextrin.

The preferred compositions for administration, particularly in oral dosage form, are the mono-, di-potassium phytate salts and mixtures thereof which may be prepared from commercially and readily available sodium phytate by initially removing the sodium using ion exchange chromatography on a suitable resin, such as Dowex beads. The free phytic acid may then be treated with potassium hydroxide to convert to the mono- and di-potassium phytate salt, with or without a dephosphorylating enzyme.

The preferred method of administration of the compositions according to the present invention is through oral administration in liquid or tablet form. As described hereinabove, the compositions may be administered as pharmaceutically acceptable salts such as salts with alkali metal cations (sodium, potassium, lithium), ammonium salts and salts with organic bases such as piperidine, triethanolamine, diethylaminoethylamine salts, and the like.

In addition to the active ingredients, the composition may also contain an effective proportion, usually from 0.001 to 0.1% weight by volume, of a pharmaceutically acceptable preservative or sterilizing agent such as cetyl pyridinium chloride, tetradecyltrimethyl ammonium bromide (commercially known as Centramide), benzyl dimethyl [2-(2-)p-(1,1,3,3-tetramethyl butyl))phenoxy)ethoxy] ammonium chloride (known commercially as Benzethonium Chloride) and myristyl-gamma-picolinium chloride.

The pharmaceutical composition may also contain conventional excipients, e.g., sodium chloride, dextrose, mannitol, and buffers such as sodium dihydrogen ortho phosphate, disodium hydrogen phosphate, sodium citrate/citric acid, and boric acid/sodium borate. The proportion and concentration of excipients and buffers may be varied within fairly wide ranges, providing the resulting solution is stable and nonirritating when administered. The preferred method of administration is by oral administration as a solid compound. The composition may be prepared in the conventional manner as tablets, pills or powders, using conventional carriers.

The phytic acid (inositol hexakisphosphate) may be combined with other inactive substances as are known in the pharmaceutical arts such as, for example, lactates, silicates, and magnesium stearate, as well as with other active substances, such as pharmaceutical drugs and vitamins, etc.

For oral administration, in a preferred embodiment, the active ingredient of the composition will also contain an enzyme such as 3-phytase (EC 3.1.38), 6-phytase (EC 3.1.3.26) or acid phosphatase which, when exposed to the digestive tract, will assist in hydrolyzing one or more of the phosphate groups from the active ingredient. Since phytic acid or phytate salts are not naturally present in animals, the digestive enzymes in animals are believed to be insufficient to completely hydrolyze the phosphate groups. Therefore, to enhance the hydrolysis of the phosphate groups in an animal or man, it is preferred that the active ingredient be administered with one or more of the aforementioned enzymes, with the preferred enzyme being 3-phytase (EC 3.1.38).

The phytic acid (inositol hexakisphosphate), phytate salt, an isomer or hydrolysate of phytic acid or a phytate salt, or a mixture of any combination thereof, with or without a dephosphorylating enzyme, can be administered in any manner as known in the art. In one embodiment the phytic acid is administered orally in encapsulated form. The oral administration can be in the form of a capsule, quick dissolve tablet, table, or other form as known in the art. The dosage of phytic acid is an amount that is pharmaceutically effective for the disease state of the patient and an amount effective as a prophylactic treatment for prevention and/or treatment of Alzheimer's Disease, and/or protein aggregation diseases and/or amyloidoses, including AL amyloidosis, amyloid A (AA) reactive systemic amyloidosis, amyloid trans-thyretin (ATTR), accumulation of islet amyloid polypeptide type 2 diabetes, and/or a neuro-degenerative disease, and/or Parkinson's Disease and/or Parkinsonism. In one embodiment the dosage is from orally about 0.5 g to about 18.75 g of phytic acid, phytate salt, an isomer or hydrolysate of phytic acid or a phytate salt, or a mixture of any combination thereof, with or without a dephosphorylating enzyme, per day.

In an alternate embodiment of about 0.5 to about 18.73 grams of phytic acid administered, dosage is at least once a week up to every day. In an alternate embodiment, dosage is every other day. In a preferred embodiment, dosage is 5 days administration, followed by 2 days off, 0.5-18.75.

It should also be understood that the dosage of once per day can mean divided doses throughout the duration of the day. For example, if the dosage is 10 gms, once a day, the dosage can be proportionately or disproportionately divided so that the 10 gms of IP6 could be taken at a dosage of 3.33 gms three times per day, or at a dosage of 4 gms, 3 gms and 3 gms per day, adding up to the daily cumulative dosage of 10 gms once a day. These divided doses during the day remain equivalent in quantity to a dosage of 10 gm once a day.

The present invention can be better understood by reference to the following examples. The following examples illustrate the present invention and are not intended to limit the invention or its scope in any manner.

### Examples

The compounds disclosed in the above referenced patents of Sabin were tested using well established standard routine testing, as known in the art, at Oregon Science and Health University in an in-vitro challenge test.

Two pages of the data of these in vitro tests are provided herein. In the first test, phytic acid attenuates APP CTF-induced neurotoxicity. APP is a precursor/source to beta amyloid plaque and is considered by many to be a “bad actor”, or “cause”, of Alzheimer's Disease. See DDN BENCH PRESS. Neither scyllo inositol nor myo inositol previously used by Barak et al, “inositol treatment of Alzheimer's disease: a double blind, cross-over placebo controlled trial”, Prog. Neuro-Psychopharmacol. & Biol. Psychiatry., 20(4): 729-735, 1996 attenuated APP CTF-induced neurotoxicity. Phytic acid is shown to be active, while scyllo inositol and myo inositol are shown to be inactive. These surprising and unexpected results with phytic acid attenuating APP CTF-induced neurotoxicity, while scyllo inositol and myo inositol fails in these same tests, suggests that phytic acid would be effective as a treatment for Alzheimer's Disease at a surprising, unexpected, non-obvious, severely reduced dose, as against the previously cited claimed dosages in the Alzheimer's Treatment Patents, especially since phytic acid proved scyllo inositol inactive in this in vitro challenge test. These results from this in vitro test demonstrate superiority over scyllo inositol and myo-inositol; and demonstrate novelty and reduction to practice for the prophylactic and therapeutic treatment of Alzheimer's disease.

In vitro studies were conducted in MC65 cells, an established human neuroblastoma line that conditionally expresses the first 17 amino-terminal residues and the 99 carboxy-terminal residues of amyloid precursor protein (APP CTF). Detectable APP CTF expression occurs within 3-4 h of tetracycline withdrawal, appearance of aggregates and initial losses of culture viability occurs at about 2 days and near complete death occurs by approximately 3 days.

### Phytic Acid Attenuates APP CTF-Induced Neurotoxicity

Phytic acid protected the cells from APP CTF-induced death in a concentration dependent manner with an EC₅₀ concentration of approximately 100 μM and concentrations of 200 μM and higher providing near complete protection from cytotoxicity. Treatment with 100 μM phytic acid suppressed oxidative stress associated with APP CTF expression, as measured by concentrations of hydrogen peroxide in the cell media.

Scyllo- and Myo-Inositol do not Rescue Cells from Ab-Induced Neurotoxicity

Stereoisomers of cyclohexanehexol-(the parent compound of phytic acid) protect primary cultured neurons from Ab oligomer-induced cytotoxicity and prevent/reverse Alzheimer phenotype in a mouse model {McLaurin, 2006#103}, {McLaurin, 2000 #104}. To test the specific of phytic acid's effects on Mc65 cells, cells were treated with the cyclohexanehexol isomers scyllo and myo-inositol at doses of 10 μM (a dose that rescues NGF-differentiated PC12 cells from Ab-induced neurotoxicity) {McLaurin, 2000 #104} and 100 mM (the EC₅₀ of phytic acid). Neither isomer rescued MC65 cells from APP CTF-induced cytotoxicity.

In vivo Test Results

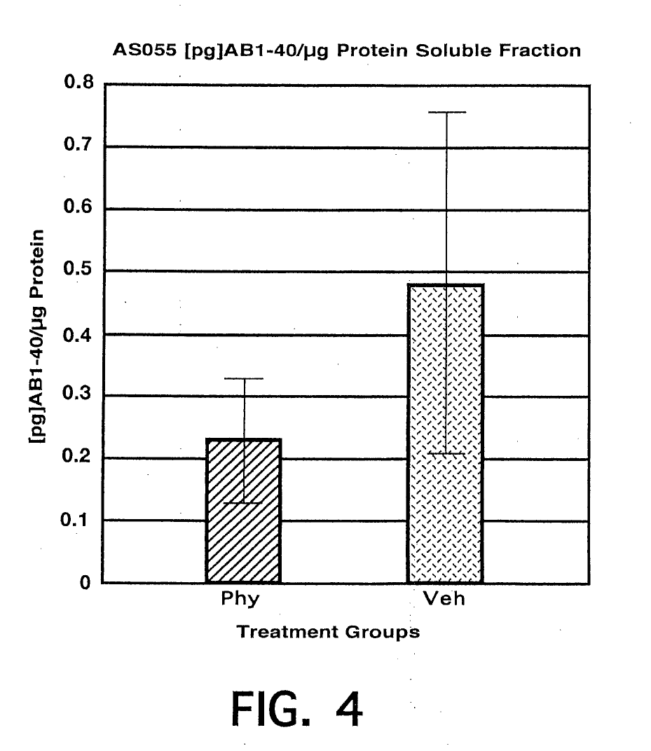

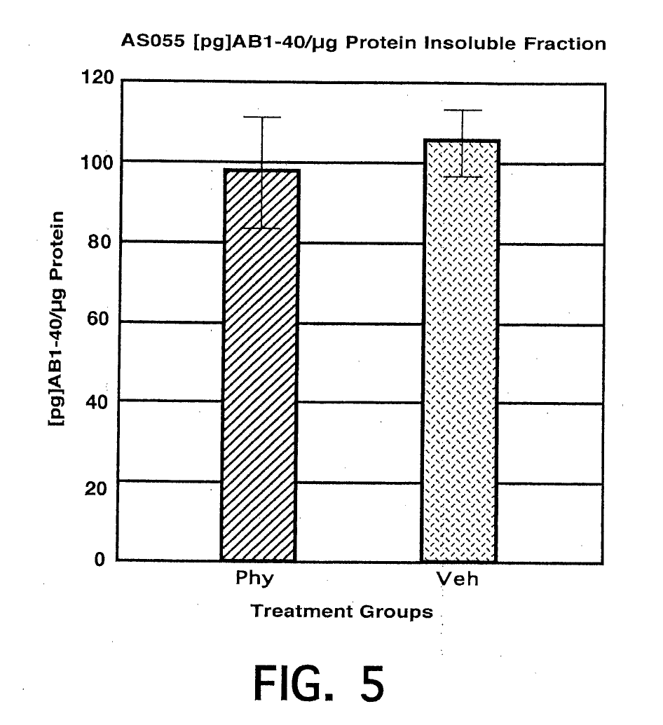

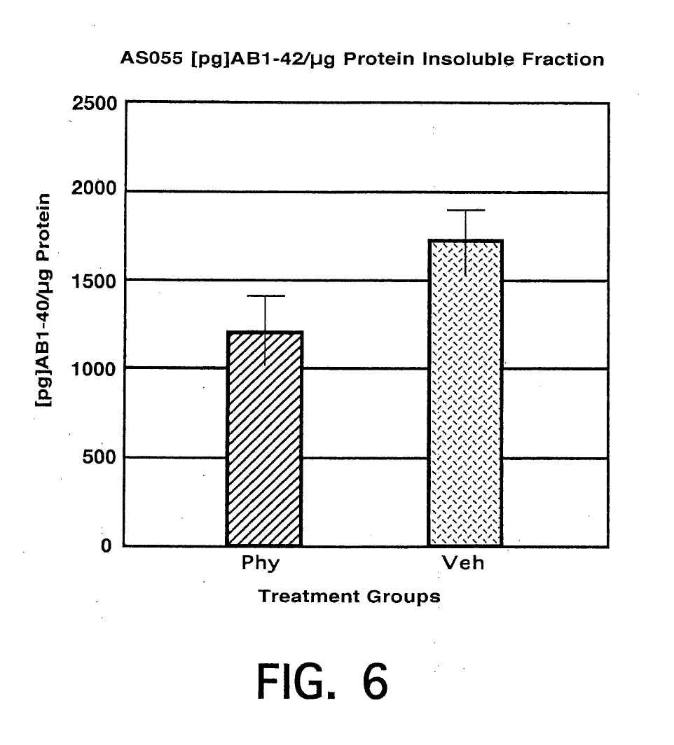

Table 1 and FIGS. 4-6 show data from in vivo testing of the subject matter compounds in Alzheimer's transgenic mice. These Alzheimer's transgenic mice are genetically altered to produce Abeta plaque, and were treated for about six months with the present inventive compounds of phytic acid. Namely, the drinking water was infused with phytic acid so that the final concentration of phytic acid in the drinking water was 2%.

After six months, the animals were put down for autopsy and necropsy so that Abeta plaque in the brains was assayed, separated and weighed.

There was no toxicity from the phytic acid in the treated group, nor any significant weight loss.

### Results

The production of two different fractions of Abeta plaque insoluble species was lowered or reduced. One species of soluble Abeta plaque was lowered or reduced. All species tested were lowered or reduced, resulting in a complete in vivo success, without toxicity.

Table 1 is a tabulation of all results. FIGS. 4-6 are graphs comparing the tested compound in vivo with the control vehicle. In all three cases, there was a reduction in Abeta plaque after treatment with 2% phytic acid, compared to the control vehicle without phytic acid.

### Key For Table 1 and FIGS. 4-6

Phy=IP6 at 2% phytic acid in mixed liquid drinking water with food
Veh=Negative control vehicle with laboratory food alone.
ID numbers=which animal was treated

TABLE 1

| | | | | AB 1-40 | | Results AB 1-40 | | AB 1-42 | | Results AB 1-42 | |
|---|---|---|---|---|---|---|---|---|---|---|---|
| ID # | Animal # | TX | Genotype | Dilution A | Dilution B | A | B | Dilution A | Dilution B | A | B |
| 1 | 4562 | Phy | app | 16 | 200 | 2320.14 | 70485.195 | 2 | 3200 | N/A | 880713.806 |
| 2 | 4563 | Phy | app | 16 | 200 | 2851.523 | 81486.942 | 2 | 3200 | N/A | 698145.624 |
| 3 | 4578 | Phy | app | 16 | 200 | N/A | 30256.773 | 2 | 3200 | N/A | 480036.365 |
| 4 | 4586 | Phy | app | 16 | 200 | 8.402 | 38495.306 | 2 | 3200 | N/A | 705439.347 |
| 5 | 4510 | Phy | app | 16 | 200 | 1219.51 | 64667.238 | 2 | 3200 | N/A | 207020.619 |
| 6 | 4831 | Phy | app | 16 | 200 | 1426.681 | 36921.948 | 2 | 3200 | N/A | 703352.803 |
| 7 | 4567 | Phy | app | 16 | 200 | 358.289 | 33040.467 | 2 | 3200 | N/A | 650661.541 |
| 8 | 4582 | Phy | app | 16 | 200 | 571.272 | 50066.589 | 2 | 3200 | N/A | 528179.357 |
| 9 | 4564 | Veh | app | 16 | 200 | 13828.275 | 92831.01 | 2 | 3200 | N/A | 1349188.224 |
| 10 | 4559 | Veh | app | 16 | 200 | 2819.85 | 55137.189 | 2 | 3200 | N/A | 800295.049 |
| 12 | 4579 | Vah | app | 16 | 200 | 19.911 | 31501.466 | 2 | 3200 | N/A | 772672.856 |
| 13 | 4581 | Veh | app | 16 | 200 | 148.018 | 44625.892 | 2 | 3200 | N/A | 983345.715 |
| 14 | 4617 | Veh | app | 16 | 200 | 1309.839 | 56358.841 | 2 | 3200 | N/A | 759029.968 |
| 15 | 4575 | Veh | app | 16 | 200 | 560.149 | 53099.902 | 2 | 3200 | N/A | 947059.623 |
| 16 | 4622 | Veh | app | 16 | 200 | 4527.412 | 62445.043 | 2 | 3200 | N/A | 659260.111 |
| 17 | 4811 C | Veh | Wt | 4 | 3200 | N/A | N/A | 2 | 800 | N/A | 53927.115 |
| 18 | 4808 C | Veh | App | 4 | 3200 | 144.004 | 1306162.415 | 2 | 800 | N/A | 166553.084 |
| 19 | 4821 C | Veh | Wt | 4 | 3200 | N/A | N/A | 2 | 800 | N/A | 90589.325 |
| 20 | 4811 H | Veh | Wt | 4 | 800 | N/A | N/A | 2 | 800 | N/A | 28596.199 |
| 21 | 4809 H | Veh | App | 4 | 800 | 91.304 | 69277.345 | 2 | 800 | N/A | 52953.772 |
| 22 | 4824 H | Veh | Wt | 4 | 800 | N/A | N/A | 2 | 800 | N/A | 27824.951 |
| 23 | 4812 C | Veh | App | 4 | 3200 | 82.825 | 975775 | 2 | 800 | N/A | 193869 |
| 24 | 4812 H | Veh | App | 4 | 800 | 48.566 | 16341 | 2 | 800 | N/A | 78.863 |
| 11 | 4361 | Veh | Wt | 16 | 200 | N/A | N/A | 2 | 3200 | N/A | N/A |

| | | | | | [ug]AB1-42 μg | |
|---|---|---|---|---|---|---|
| | BCA Results | | [ug]AB1-40 μg Protein | | Protein | |
| ID # | Fraclion A | Fraclion B | A | B | A | B |
| 1 | 5885.725 | 518.558 | 0.39413086 | 135.665681 | N/A | 1695.12125 |
| 2 | 4147.297 | 577.817 | 0.68754522 | 106.412484 | N/A | 1288.24694 |
| 3 | 2933.011 | 227.379 | N/A | 133.067579 | N/A | 2111.17282 |
| 4 | 1586.905 | 455.21 | 0.0018317 | 84.5660377 | N/A | 1549.81074 |
| 5 | 6216.961 | 491.504 | 0.2337587 | 131.570115 | N/A | 421.198238 |
| 6 | 5967.892 | 687.425 | 0.23906747 | 53.710511 | N/A | 1023.17024 |
| 7 | 6890.816 | 699.027 | 0.05354937 | 17.2663674 | N/A | 930.810313 |
| 8 | 1193.308 | 504.217 | 0.06972421 | 82.8577866 | N/A | 874.111675 |
| 9 | 8845.047 | 670.862 | 2.01989192 | 138.375717 | N/A | 2011.09651 |
| 10 | 8917.157 | 455828 | 0.40766026 | 118.338432 | N/A | 1717.63674 |
| 12 | 1740.048 | 411.584 | 0.00420059 | 83.4185811 | N/A | 1858.19105 |
| 13 | 3521.983 | 295.894 | 0.04292689 | 112.721819 | N/A | 2483.86385 |
| 14 | 6026.783 | 545.151 | 0.21720361 | 103.380936 | N/A | 1392.31445 |
| 15 | 5599.192 | 542.265 | 0.10123433 | 97.9224217 | N/A | 1746.48857 |
| 16 | 7442.038 | 723.057 | 0.60829108 | 86.3625454 | N/A | 911.768244 |
| 17 | 7002.482 | 567.048 | N/A | N/A | N/A | 94.0433879 |
| 18 | 7063.185 | 524.516 | 0.02038797 | 2490.22416 | N/A | 317.536708 |
| 19 | 5218.613 | 394.671 | N/A | N/A | N/A | 229.531242 |
| 20 | 5424.769 | 458.096 | N/A | N/A | N/A | 62.4240312 |
| 21 | 5473.254 | 354.333 | 0.01668178 | 195.514798 | N/A | 149.446345 |
| 22 | 6289.239 | 525.337 | N/A | N/A | N/A | 52.9655076 |
| 23 | 8814.989 | 515.843 | 0.01215336 | 1891.61237 | N/A | 375.829467 |
| 24 | 6047.491 | 510.065 | 0.00796492 | 32.0070933 | N/A | 154.613628 |
| 11 | 4092.036 | 332.112 | N/A | N/A | N/A | N/A |

Because of the unexpected finding that phytic acid lowers both soluble and insoluble Amyloid production in Alzheimer's transgenic mice, it is reasonable that phtyic acid (inositol hexakisphosphate) will be useful in Amyloidoses, including AL amyloidosis, amyloid A (AA) reactive systemic amyloidosis, amyloid trans-thyretin (ATTR), accumulation of islet amyloid polypeptide type 2 diabetes, and protein aggregating disease.

Furthermore, because the phytic acid (inositol hexakisphosphate) and its related compounds with or without a dephosphorylating enzyme, disclose in vitro neuroprotective activity suggesting usefulness in treatment of Alzheimer's Disease, it follows that the same compounds will be useful for treatment of motor neuron disease, or amyotrophic lateral sclerosis (ALS), in which the motor neurons unexplainably die off. The use of phytic acid and its related compounds, with or without a dephosphorylating enzyme, are also useful in treating other neuro-degenerative diseases, Parkinson's Disease and Parkinsonism.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention.

It is further known that other modifications may be made to the present invention, without departing the scope of the invention.

1. A composition for the treatment of one or more of the diseases selected from the group consisting of Alzheimer's disease, protein aggregation diseases, amyloidoses, neuro-degenerative diseases, Parkinson's Disease and Parkinsonism, comprising a pharmaceutically effective amount of at least one compound selected from the group consisting of phytic acid, inositol hexakisphosphate, a phytate salt, an isomer or hydrolysate of phytic acid or a phytate salt, and mixtures thereof, being administered to a person in an amount from about 0.5 grams to about 18.75 grams, at least once a week, with or without a dephosphorylating enzyme.

2. The composition as in claim 1 wherein the compound is ingested orally.

3. The composition as in claim 1 wherein the compound is administered topically.

4. The composition as in claim 1 wherein the compound is administered transdermally.

5. The composition as in claim 1 wherein the compound is administered intradermally.

6. The composition as in claim 1 including said dephosphorylating enzyme.

7. The composition as in claim 6 wherein said dephosphorylating enzyme is a phytase enzyme.

8. The composition as in claim 1 wherein the dosage is once a day.

9. The composition as in claim 1 wherein the dosage is every other day.

10. The composition as in claim 1 wherein the dosage is 5 days administration and then two days off per week.

11. The composition as in claim 1 wherein said dosage is predetermined and proportionately or disproportionately divided so that said dosage is administered in proportionally or disproportionately reduced amounts which cumulatively add up to said predetermined dosage, in a time period where said proportionately or disproportionately divided dosages are cumulatively equivalent in quantity to said predetermined dosage.

12. A method for the treatment of one or more of the diseases selected from the group consisting of Alzheimer's disease, protein aggregation diseases, amyloidoses, neuro-degenerative diseases, Parkinson's Disease and Parkinsonism diseases comprising the steps of administering to a person in need thereof a pharmaceutically effective amount of at least one compound selected from the group consisting of phytic acid, inositol hexakisphosphate, a phytate salt, an isomer or hydrolysate of phytic acid or a phytate salt, and mixtures thereof, being administered in an amount from about 0.5 grams to about 18.75 grams at least once per week, with or without a dephosphorylating enzyme.

13. The method as in claim 12 wherein the compound is ingested orally.

14. The method as in claim 12 wherein the compound is administered topically.

15. The method as in claim 12 wherein the compound is administered transdermally.

16. The method as in claim 12 wherein the compound is administered intradermally.

17. The method as in claim 12 wherein the dosage is once a day.

18. The method as in claim 12 wherein the dosage is every other day.

19. The method as in claim 12 wherein the dosage is 5 days administration and then two days off per week.

20. The method as in claim 12 wherein said dosage is predetermined and proportionately or disproportionately divided so that said dosage is administered in proportionally or disproportionately reduced amounts which cumulatively add up to said predetermined dosage, in a time period where said proportionately or disproportionately divided dosages are cumulatively equivalent in quantity to said predetermined dosage.

21. The method as in claim 12 wherein said neuro-degenerative diseases and protein aggregation diseases are selected from the group consisting of multiple sclerosis, conditions of the central or peripheral nervous system or systemic organ associated with a disorder in protein folding or aggregation, or amyloid formation, deposition, accumulation, or persistence; abnormal protein folding, abnormal protein aggregation, amyloid formation, deposition, accumulation, or persistence, or amyloid lipid interactions conditions causing the dissociation of abnormally aggregated proteins and/or dissolving or disrupting pre-formed or pre-deposited amyloid fibril or amyloid in a subject; amyloidoses, AL amyloidosis, amyloid A (AA) reactive systemic amyloidosis, amyloid trans-thyretin (ATTR), accumulation of islet amyloid polypeptide type 2 diabetes, conditions of the central or peripheral nervous system or systemic organ resulting in the deposition of proteins, protein fragments and peptides in beta-pleated sheats and/or fibrils and/or aggregates; amyloid angiopathy; mild cognitive impairment; Alzheimer's disease-related dementia; tauopathy; alpha.-synucleinopathy; Amyotrophic Lateral Sclerosis; motor neuron disease; Huntington's Disease, spinocerebellar ataxia, Freidrich's Ataxia; neuro-degenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy; Inclusion Body myositis due to amyloid beta peptide; Familial and Finnish Type Amyloidosis; Systemic amyloidosis associated with multiple myeloma; Familial Mediterranean Fever; chronic infections and inflammations; Type II Diabetes Mellitus associate with islet amyloid polypeptide (IAPP), vascular caused Alzheimer's Disease, Alzheimer dementia and tauopathy selected from the group of argyrophilic grain dementia, corticobasal degeneration, dementia pugilistica, diffuse neurofibrillary tangles with calcification, frontotemporal dementia with Parkinsonism, Hallervorden-Spatz disease, myotonic dystrophy, Niemann-Pick disease type C, non-Guamanian Motor Neuron disease with neurofibrillary tangles, Pick's disease, postencephalitic Parkinsonism, prion protein cerebral amyloid angiopathy, progressive subcortical gliosis, progressive supranuclear palsy, subacute sclerosing panencephalitis, tangle only dementia; alpha.-synucleinopathy selected from the group of dementia with Lewy bodies, multiple system atrophy with glial cytoplasmic inclusions, Shy-Drager syndrome, striatonigral degeneration, olivopontocerebellar atrophy, neuro-degeneration with brain iron accumulation type I, olfactory dysfunction, motor neuron disease associated with filaments and aggregates of neurofilament and/or superoxide dismutase proteins, spastic paraplegia associated with defective function of chaperones and/or triple A proteins and spinocerebellar ataxia including DRPLA or Machado-Joseph Disease; Prion related disease selected from the group of Creutzfeldt-Jakob disease, Kuru, fatal familial insomnia, Gerstmann-Straussler-Scheinker disease, and variant Creutzfeldt-Jakob disease and the amyloid polyneuropathy including senile amyloid polyneuropathy or systemic amyloidoses, Parkinson's Disease, Parkinsonism, atherosclerosis and arteriosclerosis.

22. The method as in claim 12 including said dephosphorylating enzyme.

23. The method as in claim 22 wherein said dephosphorylating enzyme is a phytase enzyme.
